(19) United States
(12) Patent Application Publication
(10) Pub. No.: US 2010/0174186 A1
(43) Pub. Date: Jul. 8, 2010

(54) METHOD AND APPARATUS FOR DETERMINING HYDRATION LEVELS BY MEASURING VELOCITY CHANGE

(75) Inventors: Eric Cohen-Solal, Ossining, NY (US); Yan S. Shi, Sleepy Hollow, NY (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/294,650

(22) PCT Filed: Mar. 29, 2007
(86) PCT No.: PCT/IB2007/051134

§ 371 Date: Sep. 26, 2008

Related U.S. Application Data
(60) Provisional application No. 60/788,452, filed on Mar. 31, 2006.

(51) Int. Cl. A61B 8/00

(52) U.S. Cl. 600/438

(57) ABSTRACT

A method and apparatus (100, 200, 400, 500) for determining patient dehydration is disclosed. The method includes measuring a velocity of mechanical waves.

Human beings and many animals rely on water to live. Water is essential to many biological and biochemical reactions that take place. As a result it is important maintain a minimum amount of water in the body.

Water is exchanged dynamically between the body and the environment. Under normal conditions, body fluids are well maintained in terms of both electrolyte concentrations and volume through processes like drinking, urine production and sweating. However, fluid balance may be disturbed due to a variety of reasons, including, but not limited to: insufficient water intake due to conditions such as chronic hypodipsia; gastrointestinal losses due to illness; renal conditions; skin losses; and clinical procedures such as hemodialysis.

All of these conditions can result in excessive fluid loss, and attendant dehydration or fluid deficit. More generally, dehydration refers to water loss with or without accompanied electrolyte loss, particularly sodium. Fluid loss of only a few percent of body weight causes discomfort and impaired body function. As dehydration levels increase, patients become fatigued and irritable, with symptoms of dry mouth, less-frequent urination and tachycardia.

Without proper water replenishment, fluid deficits can eventually develop to a clinical emergency when fluid loss is greater than 9% of body weight. Fluid deficits of such magnitudes can result in organ damage, coma, or even death.

From the above, it can be appreciated that the early identification of dehydration followed by prompt and adequate fluid intake can substantially reduce the risk of severe dehydration, and the potentially severe complications thereof.

Commonly, the clinical assessment of the level of hydration is mainly based on physical examination. Symptoms of dehydration include dry mouth and mucous membrane, sunken eyes, orthostatic hypotension, delayed capillary refill, and poor skin turgor. These symptoms are often recognized by physical examination. However, the clinical assessments can be subjective and have a low sensitivity and specificity in general.

Laboratory tests on blood and urine samples have also been used to determine dehydration status. Typically, laboratory tests are performed after physical assessment of dehydration symptoms to generate additional information; to validate the diagnosis; and to aid treatment. The main advantage of these tests over physical examination is that they provide objective and quantitative measurements. Nevertheless, they require special lab equipment and are usually time-consuming and costly.

There is a need, therefore, for a method and apparatus adapted to provide an accurate measure of hydration levels in patients that overcome at least some of the shortcomings described above.

In accordance with an example embodiment, an apparatus includes a transmitting (Tx) transducer operative to transmit a mechanical wave. The apparatus also includes a receiving (Rx) transducer operative to receive the mechanical waves transmitted from the Tx transducer, wherein the Tx transducer and the Rx transducer are disposed over a same side of a layer of tissue. The apparatus includes a processor operative to calculate a velocity of the mechanical wave in a medium, wherein the velocity is representative of a hydration level in the medium.

In accordance with another example embodiment, a method comprises: transmitting a mechanical wave at a side of a layer of tissue; receiving the mechanical waves transmitted at the side of the layer of tissue; and calculating a velocity of the mechanical wave in a medium, wherein the velocity is representative of a hydration level in the medium.

In accordance with another example embodiment, an apparatus includes a transmitting (Tx) transducer operative to transmit a mechanical wave. The apparatus also includes a reflective element operative to reflect the mechanical waves transmitted from the Tx transducer, wherein the Tx transducer and the reflective element are disposed over a same side of a layer of tissue. In addition, the apparatus includes a processor operative to calculate a velocity of the mechanical wave in a medium, wherein the velocity is representative of a hydration level in the medium.

In accordance with yet another example embodiment, a method, comprises: transmitting transmit a mechanical wave at a side of a layer of tissue; reflecting the mechanical wave transmitted at the side of the layer of tissue; and calculating a velocity of the mechanical wave in a medium, wherein the velocity is representative of a hydration level in the medium.

As used herein, the terms ‘a’ and ‘an’ mean one or more; and the term ‘plurality’ means two or more.

As used herein, the term ‘patient’ includes humans, mammals and fish.

The invention is best understood from the following detailed description when read with the accompanying drawing figures. It is emphasized that the various features are not necessarily drawn to scale. In fact, the dimensions may be arbitrarily increased or decreased for clarity of discussion.

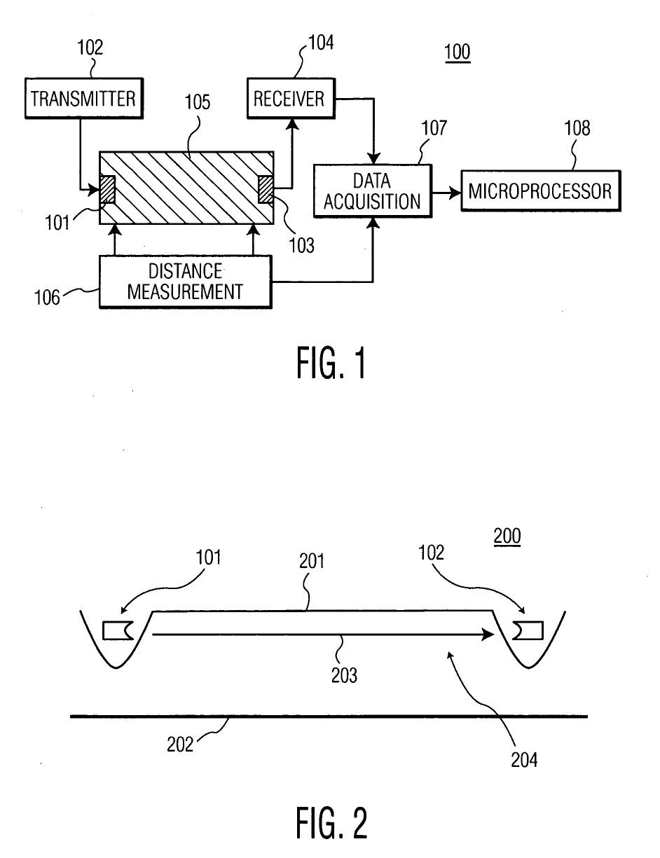

FIG. 1 is a simplified block diagram of an apparatus in accordance with an example embodiment.

FIG. 2 is a cross-sectional view a dehydration sensor contacting the surface of a body in accordance with an example embodiment.

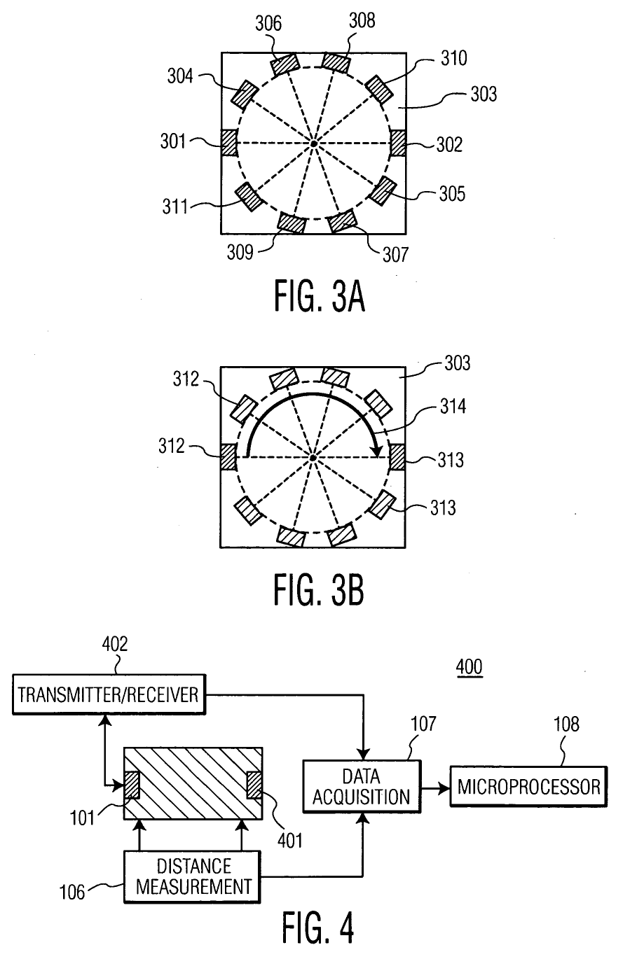

FIG. 3A is a top-view of a dehydration sensor in accordance with an example embodiment.

FIG. 3B is a top-view of a dehydration sensor in accordance with an example embodiment.

FIG. 4 is a cross-sectional view a dehydration sensor contacting the surface of a body in accordance with an example embodiment.

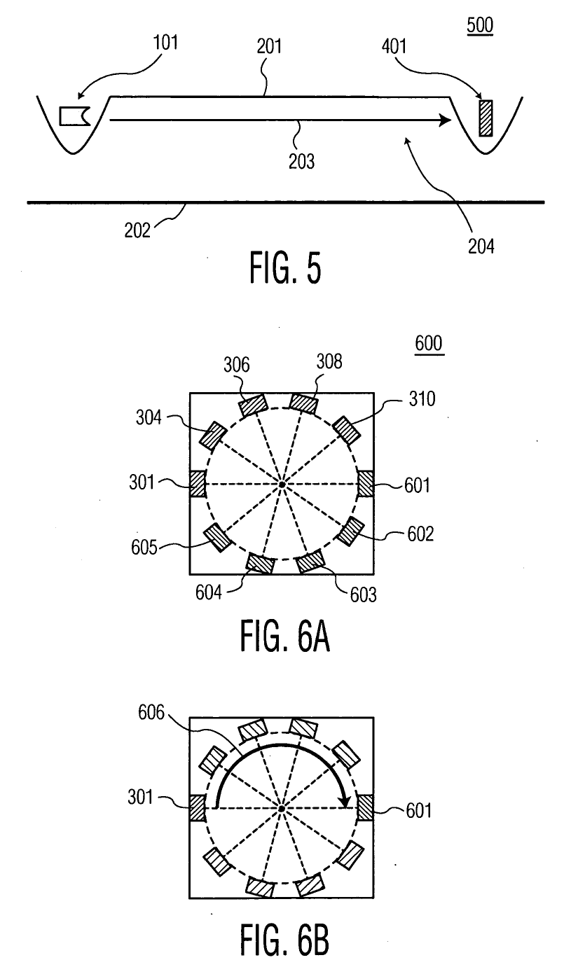

FIG. 5 is a simplified schematic representation of an apparatus in accordance with an example embodiment.

FIG. 6A is a top-view of a dehydration sensor in accordance with an example embodiment.

FIG. 6B is a top-view of a dehydration sensor in accordance with an example embodiment.

In the following detailed description, for purposes of explanation and not limitation, example embodiments disclosing specific details are set forth in order to provide a thorough understanding of the present teachings. However, it will be apparent to one having ordinary skill in the art having had the benefit of the present disclosure that other embodiments that depart from the specific details disclosed herein. Moreover, descriptions of well-known devices, hardware, software, firmware, methods and systems may be omitted so as to avoid obscuring the description of the example embodiments. Nonetheless, such hardware, software, firmware, devices, methods and systems that are within the purview of one of ordinary skill in the art may be used in accordance with the example embodiments. Finally, wherever practical, like reference numerals refer to like features.

FIG. 1 is a simplified schematic block diagram of a dehydration measurement apparatus 100 in accordance with an example embodiment. The apparatus 100 includes a transmitting (Tx) transducer 101 connected to a transmitter 102; and a receiving (Rx) transducer 103 is connected to a receiver 104. The transducers 101, 103 are disposed over or on a structure 105 made of a material that will not interfere with the mechanical or electrical properties of the transducers 102, 103. In a specific embodiment, the structure 105 is semi-circular in shape with the transducers at specific locations of the arc of the semi-circle. This allows the transducers 101, 103 to be pressed into place for measurement, while accommodating the portion of the body being tested and not interfering with the measurement.

The apparatus 100 also includes a distance measurement device 106, a data acquisition module 107 and a processor 108.

The distance measurement device 106 measures the distance between the transducers 102, 103 so that the velocity of mechanical waves can be determined. The distance measurement device 106 may be a precision mechanical caliper, or a precision laser-based caliper, both of which are within the purview of one of ordinary skill in the measurement and testing arts. In a specific embodiment, the caliper is a vernier caliper with digital readouts. Illustratively, the accuracy of the caliper is approximately 0.1% or less of the measuring distance. Such accuracy is well within the limits of commercially available calipers.

The transmitter 102 transmits electrical pulses of finite duration and at a chosen periodicity to the transducer 101. The electrical pulses are converted to mechanical pulses/waves that are emitted by the transducer 101 into the body. As it is beneficial to provide for a plurality of velocity measurements in a single test, multiple pulses are generated at the transmitter and converted to mechanical waves and transmitted by the Tx transducer 101. As such, when a test is begun the transmitter 102 will transmit the short duration pulses periodically, with each pulse providing a measurement of the velocity. In an example embodiment, more than five pulses are provided to exact a measurement.

The electrical pulses from the transmitter 102 are relatively short in duration and have a suitable amplitude so that when converted to a mechanical wave, attenuation of the mechanical waves in the region between the Tx transducer 101 and the Rx transducer 103 may be accommodated. To this end, when a velocity measurement according to an embodiment is carried out using mechanical waves at typical ultrasonic frequencies, the transmitter 102 usefully provides sub-microsecond pulses having sufficient amplitudes to facilitate signal processing at the receiver 104. Illustratively, the electrical signals from the transmitter 102 are on the order of approximately 10² V.

The mechanical waves transmitted from the Tx transducer 101 propagate through the body and are received at the Rx transducer 103 (also referred to as a pickup transducer). These mechanical waves are converted into electrical signals by the Rx transducer 103 and transmitted to the receiver 104. The receiver 104 includes a receiver circuit, filters and amplifiers. The amplifiers are useful particularly when the mechanical waves are relatively high frequency, as the attenuation of the mechanical wave in the layers of the human body increases with increasing frequency. Filtering is useful in reducing noise before and after amplification of the electrical signal. Suitable receiver circuits, filter circuits and amplifier circuits to realize the receiver 104 are well known to those skilled in analog signal processing.

The receiver 104 provides the electrical data from the received pulses to a data acquisition module 107. The module 107 illustratively includes a register or memory adapted to store signal/waveform data received from the receiver 104. These data are then retrieved/captured for calculating the velocity at the processor 106.

The module 107 also receives a distance measurement from the distance measurement device 106. These data are coupled with the waveform data for each measurement in the register or memory so that a velocity may be ascertained for each waveform.

The processor/microprocessor 108 retrieves the data from a series of measurements and is provided to effect various functions of the apparatus 100. The processor 108 may be a commercially available microprocessor such as a Pentium® from Intel Corporation, or other suitable processor. The processor 108 optionally includes operating system (OS) software and application code written to effect the algorithms described herein. Such code is within the purview of one of ordinary skill in the art.

In an example embodiment, the processor 108 is adapted to implement data capture and carry out correlation algorithm. Notably, the data capture includes analog to digital (A/D) conversion of received electrical signals, and storage of the data. The time of flight is determined based on the correlation algorithm. Alternatively, the time of flight may be determined via edge detection of the reflected signal, such as positive/negative slope, zero-crossing techniques known in the art.

Once the time of flight is determined for a particular waveform, the processor calculates the velocity by dividing the distance by the time of flight. Notably, the velocity of sound can vary linearly with the temperature of the tissue or other layer of the body. For example, in muscle tissue, the velocity will increase/decrease approximately 1.1 m/s-° C. to approximately 1.2 m/s-° C. In blood the velocity of the mechanical waves will vary by approximately 2.0 m/s-° C. In skin the velocity varies by approximately 0.1% ° C. As such, the processor 108 may be configured to algorithmically correct the calculated speed to account for the temperature.

In an example embodiment, a baseline velocity is determined at a particular temperature. Next, a thermo-couple or other suitable thermometer is used to determine the temperature of the tissue or substance in which the velocity is being tested. The mechanical waves are then into the tissue or substance. After testing is completed and the raw velocity determined, the processor 108 algorithmically adjusts the velocity measured, providing a calibrated velocity for each measurement.

In operation, upon receiving an input from an operator, the transmitter 102 triggers the transmission of pulses by the Tx transducer 101 to begin a test. A plurality of transmissions is executed in sequence in order to provide a plurality of measurements at a particular location of the body. In addition, one or more measurements may be made at one or more addition locations of the body. These data can then be compiled at the data acquisition module along with the temperature data for each measurement and the velocity for each measurement can be calculated by the processor 106. The processor may then calculate an average velocity for each location. In addition, a composite average velocity may be calculated from the average velocity from each location.

As noted, many measurements from transmitted and received pulses may be transmitted at the same location and from these an average of the velocity may be determined. In a specific embodiment, more than five measurements are used to calculate an average. In another specific embodiment, more than five measurements at each of a number of locations are used to calculate an average for each location and a composite average. The average values may also be stored in the data acquisition module 202.

After calculating the velocity or an average velocity as described above, the processor 108 algorithmically compares the velocity or the average velocity of the most recent measurement with a baseline value of the velocity. From these comparisons, a relative measure of hydration levels can be made. Alternatively, the processor 108 may algorithmically determine the fluid hydration level of a patient. As noted, the patient may be a human being, and the apparatus is a medical testing device. It is contemplated that the apparatus be used in veterinary testing of animals where concerns about dehydration require a measure of hydration levels.

The baseline value of the velocity at suitable hydration at each location for each patient may be calculated using the transducers 102, 103. Beneficially, the baseline is determined when subjects are well hydrated through drinking water or other fluids, or by intravenous fluid injection. The quantity of fluid may be recommended by nutritionists or other medical practioners under normal circumstances. In a specific embodiment, the baseline measurements are carried out over a preset number of days to ensure its reliability. The average of all measurements may then be used as a baseline.

Alternatively or additionally, the baseline turgor values may be garnered from data from a large group of patients. These population-based baselines can be further compiled demographically so that a particular patient's hydration levels can be compared to acceptable fluid levels of people of similar height, weight, age and other similar criteria.

The measurement data stored in the module 107 can be compared to the baseline value for a determination of the level of dehydration in the patient. In an example embodiment, the measurement data from multiple locations may be used to determine the dehydration level. Moreover, average velocity measurements described previously may be used to determine the dehydration level.

In accordance with an example embodiment, multiple measurements are performed in order to compile a database for the relationship between changes in the velocity of mechanical waves in a particular tissue in the body and dehydration levels for a patient. These data can then be used to map the measurements to the fluid hydration level in the patient.

FIG. 2 is a cross-sectional view of an apparatus 200 in accordance with an example embodiment. The apparatus 200 includes the Tx transducer 101 and the Rx transducer 103 described previously.

Notably, the transducers 101, 103 are disposed in a linearly separated relationship. In the present example embodiment, this is straightforward; however, in other embodiments described herein a plurality of Tx transducers and Rx transducers are included in the apparatus, and each Tx transducer must be associated with one Rx transducer and separated in a linear manner to ensure accurate data gathering. In order to ensure distance accuracy, the transducers 101, 103 are spaced approximately 1.0 cm to approximately 2.0 cm apart. Arranging the transducers 101, 103 relatively far apart is not problematic because at the frequencies of propagation, attenuation is minimal.

The transducers 102, 103 are single element transducers that operate in a pulsed mode. Accordingly, a relatively wide bandwidth, greater than approximately 25% is desirable. The transducers 102, 103 are illustratively ultrasonic transducers that emit/receive mechanical waves. In a specific embodiment, the transducers 102, 103 are unfocused. Alternatively, the transducers 102, 103 are focused transducers.

In accordance with an example embodiment, the transducers 102, 103 have a frequency illustratively in the range of approximately 0.3 MHz to approximately 10.0 MHz, or greater. Notably, the center frequency of the transducers 102, 103 is not critical and the dispersion of the mechanical waves is not significant over the illustrative frequency range. As will be appreciated by one skilled in the art, minimizing dispersion provides a greater measure of the group velocity of the mechanical waves.

As is known, the attenuation of mechanical waves in tissue will increase with increased frequency. As such it may be useful to operate the transducers 102, 103 in the range of approximately 0.3 MHz to approximately 5.0 MHz. Thereby, a greater signal amplitude at the Rx transducer 103 for a given input signal amplitude may be attained facilitating measurements and accurate velocity data.

In an illustrative embodiment, the transducers 102, 103 are pressed against a layer of tissue 201 and into another layer of tissue 204. However, the transducers 102, 103 are located away from an opposing layer of tissue 202 (e.g., skin). In a specific embodiment, the apparatus 200 is inserted into the patient's mouth and the transducers 102, 103 contact the interior surface of the mouth (in this case tissue 201). The Tx transducer 102 then transmits sound wave 203 across the tissue 204 in order for a sound velocity measurement to be made. Notably, the transducers 102, 103 are at substantially the same depth so that the tissue consistency is similar.

In a specific embodiment, the tissue 201 is the stratum corneum layer of the patient. This layer comprises a layer of ‘dead’ skin having a thickness of approximately 0.010 mm to approximately 0.020 mm. The transducers 101, 103 may then protrude into the tissue 204, which is the dermis. As is known, the level of hydration of the body is commensurate with the level of hydration of the dermis. As such, by measuring the velocity of the mechanical waves in the dermis, an accurate measure of the hydration level of the patient can be attained.

In another specific embodiment, the transducers 101, 103 are disposed on the exterior of the mouth. For example, the transducers 101, 103 are pressed into the cheek or the mandibular muscles. Then the measurement is carried out as described previously.

In yet another specific embodiment, the velocity of the mechanical waves is determined in blood. To this end, when dehydration occurs, water is frequently lost from intravascular fluids. As a result, blood tends to become more concentrated and viscous. The increase in concentration of erythrocytes in blood is proportional to blood volume reduction, provided that only water and/or electrolytes are removed from the system. This condition is satisfied in most dehydration cases. As is known, there is a direct proportionality between the sound velocity in blood and the total protein concentration (TPC), which is the sum of plasma proteins and hemoglobin. As such, as dehydration occurs, the blood becomes more viscous, and the velocity increases.

Illustratively, in order to determine the dehydration from the TPC values, the apparatus is positioned over a visible vein. The transducers 102, 103 are pressed against the skin (e.g., layer 201) and into tissue 204, which is blood in this case. Velocity measurements may be made according to the present teachings and from these measurements, the level of dehydration may be determined.

Illustratively, a plurality of measurements may be made at a particular location and the average velocity may then be calculated at the processor and compared to a baseline velocity for each location. These data can then be evaluated algorithmically at the processor 108 to determine the relative level of dehydration.

It is known that the velocity of mechanical waves in a tissue layer may depend on the orientation of tissue layer relative to the propagation direction of the waves. For example, transmitting the sound wave 203 in the tissue 204 along one orientation may provide one velocity, while rotating the transducers 102, 103 in perpendicular direction to this orientation may provide another velocity measurement. This is particularly true in tissue, such as muscle tissue, that has direction dependent orientations (e.g., muscle fibers are often oriented along one direction). Accordingly, a plurality of measurements may be made at each of a plurality of orientations to provide a more accurate velocity value for the particular location. The average velocity may then be calculated at the processor 108 and compared to a baseline velocity for each location. These data can then be evaluated algorithmically at the processor 108 to determine the relative level of dehydration.

In other example embodiments, measurements are made in a plurality of locations. Illustratively, a plurality of measurement may be made at a plurality of locations. In addition, multiple measurements at each of a plurality of transducer orientations may be made at each of these locations. The average velocity may then be calculated for each location at the processor 108 and compared to a baseline velocity for each location. These data can then be evaluated algorithmically at the processor 108 to determine the relative level of dehydration.

As can be appreciated from a review of FIG. 2, the pressure applied to the apparatus 200 governs the depth of the transducers in the tissue 201. To measure velocity in a particular layer of tissue (e.g., tissue 204) a particular pressure is required. Moreover, from one measurement to the next, in order to provide consistent measurements of a particular tissue, the depth of the transducers 102, 103 should be substantially the same. Accordingly, it is useful to provide a pressure sensor or other device to ensure that the transducers are at substantially the same depth from measurement to measurement. In a specific embodiment, a pressure sensor is provided at the transducers 102, 103 or on the apparatus 200 (e.g., on the substrate 105) to ensure that the same pressure is applied to each location of a measurement. This pressure may be from the weight of the apparatus 200 or from the weight plus an external applied force.

FIGS. 3A is a top view of an apparatus 300 in accordance with an example embodiment. The apparatus 300 includes many common features to the apparati described in connection with FIGS. 1 and 2. As such, many of these details may not be repeated to avoid obscuring the description of the example embodiments.

The apparatus 300 includes a plurality of transducers arranged in a substantially circular fashion. The number of transducers shown is merely illustrative and more or fewer transducers may be used.

Each of the transducers is located diametrically opposite to one other transducer. In the embodiment shown, transducers are paired diametrically. As such, transducer 301, 304, 306, 308, 310 are paired with transducers 302, 305, 307, 309 and 311, respectively. One of these diametrically opposed transducers is the Tx transducer and the other is the Rx transducer. For example transducer 301 is diametrically opposite to transducer 302, and illustratively transducer 301 is the Tx transducer and transducer 302 is the Rx transducer. The Tx transducer transmits a mechanical wave (shown as a dotted line) to the Rx transducer. In the example embodiment, the transducers are disposed on a circular-shaped structure and are pressed into the tissue 303 (e.g., skin) of the patient.

Usefully, the plurality of pairs of transducers allow for measurements to be taken at different orientations at a particular location without having to move a transducer pair (e.g., transducers 101, 102 of apparatus 200) repetitively. Beneficially, the apparatus 301 allows multiple measurements at each of a number of orientations. Moreover, the apparatus 300 may be moved to a number of locations and similar measurements made at each of these locations. As noted, in a specific embodiment, the transducers are arranged in a substantially circular arrangement. Therefore, all transducers are disposed on a circle so that only the diameter of the circle need to be measured to record the distance between the transducers.

In accordance with an example embodiment, a transmitter (e.g., transmitter 101) and a receiver (e.g., receiver 102) are connected to a respective Tx transducer and an Rx transducer pair. The transducers are excited as described previously and the data are provided to the data acquisition module 107. A controller (not shown) may be used to provide the sequence of measurements from one pair of transducers to another. Illustratively, the controller includes a switching element that sequentially provides pulses to the transmit transducers 301, 304, 306, 308 and 310. Such a controller may be implemented in hardware and software within the purview of one of ordinary skill in the art.

After measurements are taken, an average velocity may be determined at the processor 108 and compared by the processor 108 to a baseline value to determine the level of dehydration in the patient. If multiple locations are probed, the average velocities may be determined and the analysis of the dehydration level may be made in a similar manner to that described previously.

The apparatus of the example embodiment shown in FIG. 3B illustrates a method of obtaining multiple measurements in various orientations using two transducers. Many of the details of the transducer 300 are common to those described in connection with the example embodiments of FIGS. 1-3A and are thus not repeated.

The transducer 312 is a transmit transducer and is arranged diametrically opposite to transducer 313, which is the receive transducer. After measurements are made to determine the velocity of the tissue/substance at the first position, the transducers 312, 313 are rotated along the circumference of a circle as shown by 314 and the next measurement is made. With each rotation, the diametrically opposed transducers 312, 313 garner measurements of the velocity and thus hydration levels as described above.

FIGS. 4-6B relate to alternative embodiments. The apparati and methods described in conjunction with FIGS. 4-6B share features, methods and characteristics with the embodiments described in connection with FIGS. 1-3B. As such, many of these details may not be repeated to avoid obscuring the description of the example embodiments.

FIG. 4 is a schematic block diagram of a dehydration measurement apparatus 400 in accordance with an example embodiment. The apparatus 401 includes the Tx transducer 101 and a reflective element 401. The reflective element 401 is illustratively a known reflector used to efficiently reflect mechanical waves. A transmitter/receiver (transceiver) 402 provides the electrical pulse to the transducer 101 and receives the electrical signals representative of the reflected mechanical waves from the transducer 101. Illustratively, the transceiver 402 is combined with an electronic switch to switch mode between Tx and Rx. The transceiver 402 is a device known to those skilled in the art.

The transmitting transducer 101 functions in pulse-echo mode (Tx-Rx) and the mechanical waves travel round trip. The general sequence is that electrical pulses are provided by the transceiver 402 to the transducer 101. The transducer 101 transmit mechanical waves through tissue. The mechanical waves are reflected back from the reflective element 401 and then received by the same transducer 101. The travel distance will be round trip, or double the distance measured by the measurement device 106.

After measurements are made, the data are stored in the data acquisition module 107. The data are retrieved by the microprocessor 108, which functions in substantially the same manner as described previously. Like other embodiments, multiple measurements may be made at a plurality of locations and adjustments may be made to account for temperature.

FIG. 5 is a cross-sectional view of an apparatus 500 in accordance with an example embodiment. The apparatus 500 includes the Tx transducer 101 and the reflector 401 described previously.

In an illustrative embodiment, the transducer 101 and reflective element 401 are pressed against layer 201, into layer 204; but away from an opposing layer of tissue 202. In a specific embodiment, the apparatus 500 is inserted into the patient's mouth and the transducer 101 and the reflector 401 contact the interior surface of the mouth (in this case tissue 201). The Tx transducer 101 then transmits mechanical waves 203 across tissue 204 in order for a velocity measurement to be made. The reflective element 401 reflects the mechanical waves 203. The reflected waves traverse the tissue again and are received at the transducer 101.

In ways described previously, the velocity of the mechanical wave is calculated and the hydration level is determined.

FIG. 6A is a top view of 600 in accordance with an example embodiment. The apparatus 600 includes a plurality of transducer and diametrically opposed reflective elements disposed in a substantially circular fashion. The number of transducers and reflective shown is merely illustrative and more or fewer transducers and reflective elements may be used.

In the embodiment shown, transducers 301, 304, 306, 308, 310 are paired with reflective elements 601, 602, 603, 604 and 605, respectively. The transducers transmit mechanical waves (shown as dotted lines) to their respective reflective elements. The reflective elements reflect the mechanical waves back to their respective transducer.

After the reflective wave is received at the transducer, the velocity of the wave is calculated as described previously. Like other embodiments, multiple measurements may be made at each transducer/reflector pair and adjustments may be made to account for temperature.

The apparatus of the example embodiment shown in FIG. 3B illustrates a method of obtaining multiple measurements in various orientations using one transducer and one reflective element. Many of the details of the apparatus presently described are common to those described in connection with the example embodiments of FIGS. 4-6A and are thus not repeated.

The transducer 301 is a transmit transducer and is arranged diametrically opposite the reflective element 601. After measurements are made to determine the velocity of the tissue/substance at the first position, the transducer 301 and reflective element 601 are rotated along the circumference of a circle as shown by 606 and the next measurement is made. With each rotation, the diametrically opposed transducer and reflector garner measurements of the velocity and thus hydration levels as described above. Like other embodiments, multiple measurements may be made at each transducer/reflector pair and adjustments may be made to account for temperature.

In view of this disclosure it is noted that the various methods and devices described herein can be implemented in hardware and software. Further, the various methods and parameters are included by way of example only and not in any limiting sense. In view of this disclosure, those skilled in the art can implement the present teachings in determining their own techniques and needed equipment to effect these techniques, while remaining within the scope of the appended claims.

1. An apparatus, comprising:
a transmitting (Tx) transducer (101) operative to transmit a mechanical wave;
a receiving (Rx) transducer (103) operative to receive the mechanical waves transmitted from the Tx transducer, wherein the Tx transducer and the Rx transducer are disposed over a same side of a layer of tissue (201);
a processor (108) operative to calculate a velocity of the mechanical wave in a medium, wherein the velocity is representative of a hydration level in the medium.

2. An apparatus as recited in claim 1, wherein the Tx transducer is adapted to transmit a plurality of mechanical waves and the velocity is an average of the velocity of each of the mechanical waves.

3. An apparatus as recited in claim 1, further comprising at least one other Tx transducer (301, 304, 306, 308, 310) and at least one other Rx transducer (302, 305, 307, 309, 311), wherein each Tx transducer is linearly opposed to a respective one of the Rx transducers.

4. An apparatus as recited in claim 1, further comprising a plurality of Tx transducers and a plurality of Rx transducers arranged in a circle, wherein each of the Tx transducers is coupled in a diametrically opposed relation to a respective one of the plurality of Rx transducers.

5. An apparatus as recited in claim 1, wherein the mechanical waves are ultrasonic waves.

6. An apparatus as recited in claim 3, wherein the plurality of calculations from each of the transducers are from more than one location on a body.

7. An apparatus as recited in claim 1, wherein the medium is a dermis layer (204).

8. An apparatus as recited in claim 1, wherein the medium is intravenous blood (204).

9. An apparatus as recited in claim 1, further comprising a distance measurement device (106) adapted to measure a distance between the Tx transducer and the Rx transducer.

10. An apparatus as recited in claim 1, further comprising a temperature sensor adapted to measure a temperature of the layer of tissue.

11. A method, comprising:
transmitting mechanical waves (203) at a side of a layer of tissue (201);
receiving the mechanical waves (203) transmitted at the side of the layer of tissue; and
calculating a velocity of the mechanical wave in a medium, wherein the velocity is representative of a hydration level in the medium.

12. A method as recited in claim 11, further comprising repeating the transmitting, receiving and calculating multiple times; and calculating an average velocity.

13. A method as recited in claim 11, further comprising repeating the transmitting at more than one location of a body.

14. A method as recited in claim 11, further comprising repeating the transmitting, receiving and calculating multiple times at each of the locations; and calculating an average velocity for each of the locations.

15. An apparatus, comprising:
a transmitting (Tx) transducer (101) operative to transmit a mechanical wave;
a reflective element (401) operative to reflect the mechanical waves transmitted from the Tx transducer, wherein the Tx transducer and the reflective element are disposed over a same side of a layer (201) of tissue;
a processor (108) operative to calculate a velocity of the mechanical wave in a medium, wherein the velocity is representative of a hydration level in the medium.

16. An apparatus as recited in claim 15, wherein the Tx transducer is adapted to transmit a plurality of mechanical waves and the velocity is an average of the velocity of each of the mechanical waves.

17. An apparatus as recited in claim 15, further comprising at least one other Tx transducer (301, 304, 306, 308, 310) and at least one other reflective element (601, 602, 603, 604, 605), wherein each Tx transducer is linearly opposed to a respective one of the reflective elements.

18. An apparatus as recited in claim 15, further comprising a plurality of Tx transducers and a plurality of reflective elements arranged in a circle, wherein each of the Tx transducers is coupled in a diametrically opposed relation to a respective one of the plurality of reflective elements.

19. An apparatus as recited in claim 15, wherein the mechanical waves are ultrasonic waves.

20. An apparatus as recited in claim 3, wherein the plurality of calculations from each of the transducers are from more than one location on a body.

21. A method, comprising:
transmitting transmit a mechanical wave (203) at a side of a layer of tissue (201);
reflecting the mechanical waves (203) transmitted at the side of the layer of tissue; and
calculating a velocity of the mechanical wave in a medium, wherein the velocity is representative of a hydration level in the medium.

22. A method as recited in claim 11, further comprising repeating the transmitting, the reflecting and the calculating multiple times; and calculating an average velocity.
